(19) United States
(12) Patent Application Publication
(10) Pub. No.: US 2014/0213990 A1
(43) Pub. Date: Jul. 31, 2014

(54) COMPOSITIONS AND METHODS FOR TREATING SURFACE WOUNDS

(71) Applicant: Boris Gorinshteyn, Roswell, GA (US)

(72) Inventor: Boris Gorinshteyn, Roswell, GA (US)

(21) Appl. No.: 14/166,877

(22) Filed: Jan. 29, 2014

Related U.S. Application Data
(60) Provisional application No. 61/849,590, filed on Jan. 30, 2013.

(51) Int. Cl. A61M 35/00; A61K 36/258; A61K 36/886; A61K 45/06; A61K 31/4166; A61K 38/39; A61K 33/00; A61K 36/71; A61K 33/40

(52) U.S. Cl. CPC A61M 35/003; A61K 36/71; A61K 36/258; A61K 36/886; A61K 33/40; A61K 31/4166; A61K 38/39; A61K 33/00; A61K 45/06
USPC 604/304; 604/310; 424/726; 424/616

(57) ABSTRACT

The invention provides synergistic compositions and methods to prevent and treat dermal wounds and topical ulcers rapidly, efficaciously and economically. It comprises a synergistic combination of natural products, specifically the combination of an extract of Hydrastis canadensis, an extract of a species of the genus Panax and an extract of a Aloe barabadensis L. that are essentially free of extract of Passiflora incarnata L. The combination can be administered to wounded skin and or a wound topical cavity of a patient as a prophylactic or therapeutic dose, and may optionally be delivered by means of a wetted dressing. The invention further provides kits that may be used for topical application of the combination.

### FEDERAL GRANTS

No funds from federal sources were used in the development of this invention.

### FIELD OF THE INVENTION

The invention pertains to compositions and methods for healing topical wounds and preventing their formation.

### BACKGROUND

Flesh wounds are widely but mistakenly regarded as trivial. Currently, for 75% of otherwise healthy individuals a modest bedsore (i.e., Stage II) requires 8 weeks of diligent care. For stage IV sores (the most advanced stage) 38% never heal; the rest usually require at least a year of care. D. R. Thomas, et al., J. Am. Med. Dir. Assoc., 6(1):46-49 (2005). Pressure sores are common: the incidence is 0.4-38% in acute care, 2.2-23.9% in long-term care, and up to 29% in home care. NPUAP Board of Directors, “Pressure ulcers in America: prevalence, incidence, and implications for the future,” in An Executive Summary of the NPUAP Monograph, ed. Cuddigan, J., et al., (Jul/August 2001). Indeed at death 24% of Americans have at least one stage II or worse pressure ulcer. (1989 Nat. Funeral Dir. Assoc. (NFDA), cited in K. L. Eckman, Decubitus, 1989; 2:36-40) Diabetics, who comprise 7% of the population, are at even greater risk: they account for 60% of foot amputations, 85% of which are preceded by a foot ulcer; annually 5% of diabetics develop a foot ulcer and 1% of diabetics require a foot amputation. Cuts and burns are more acute than topical ulcers but have comparable effects. Even superficial second degree burns require at least two to three weeks to heal; third and fourth degree burns require much longer and commonly lead to disfigurement, disability, gangrene, amputation and death.

Although the shallowness of surface wounds facilitates antibiotic treatment it does not facilitate restoration. Moreover the U.S. Food and Drug Administration has approved just a few over-the-counter ingredients for wound care claims, and even those often fail to provide appreciable healing though they keep the wound aseptic. The etiology illustrates the reasons.

Patients confined to beds and wheelchairs may have pressure sores, usually at the heels, ankles, hips, buttocks, back, elbows, shoulders, or back of head. First, bone compresses tissue on a surface; the reduced perfusion can cause ischemia or necrosis in immunocompromised patients. Then deep fascia and skeletal muscle slide due to motion or gravity; the prolonged shear force pinches blood vessels. Finally, friction sheds skin layers. Moisture from incontinence, perspiration or exudates also impairs adhesion between epithelial cells, allowing maceration of skin. Old age, malnutrition, vascular disease, and smoking also have effects. Complications can be dangerous, painful and costly to treat: autonomic dysreflexia, bladder distension, osteomyelitis, pyarthroses, sepsis, amyloidosis, anemia, urethral fistula, gangrene, and malignant transformation. Patient noncompliance may result in seromas, hematomas, infections, and dehiscence. Renal failure and amyloidosis are commonly fatal. Niezgoda, J. A., S. Mendez-Eastman, Adv. Skin & Wound Care: J. Prevent. Healing, 19(1-Supp.):3-15 (2006). Brem, B., Kirsner, R. S., and Falanga, V., Am. J. Surgery, 188(1 Supp. 1):1-8 (July 2004).

Diabetes heightens the tendency. Diabetic sores often include foot ulcers, pressure ulcers, thrush, necrobiosis, bullae and peptic ulcers, and are so persistent that they rank among the three major categories of chronic wounds. Diabetes increases susceptibility due to: poor circulation (oxygen-deficiency of damaged cells); glucose-associated neuropathy (peripheral, autonomic, focal and proxima); and compromised immune systems (high vulnerability to infection). Hence many diabetic patients feel no pain from their wounds (i.e., diabetes, like Hansen's disease [leprosy] inhibits nociception). (See Snyder, R. J., Clin. Dermatol. 23 (4): 388-395, 2005). Thus the first injury is often ignored and repeatedly reinjured without notice.

Like decubitus sores, diabetic sores are an especially high priority due to demographic size. According to the National Institutes of Health, 8.3% of the US population is diagnosed with diabetes and another 3.1% have it but are undiagnosed. And each diabetic person has a 15% likelihood of developing a topical ulcer. See: Frykberg, R. G., et al., “Diabetic foot disorders: a clinical practice guideline,” J Foot and Ankle Surg., 45: S2-66, 2006; Paiumbo, P. J., Melton, L. J., “Peripheral vascular disease and diabetes,” in Diabetes In America, Harris, M. I. and Hamman, R. F., ed., NIH, Bethesda, 1985, p. 1-21; and Reiber, G. E., Boyko, E. J., Smith, D. G., “Lower extremity foot ulcers and amputations in diabetes,” in Diabetes in America, 2^{nd} ed, p. 409-427 , Harris, M. I., Cowie, C. and Stern, M. P., ed., NIH Pub. No. 95-1468; 1995.

Pressure ulcers (decuhiti) are diagnosed in six stages, though the U.S. National Pressure Ulcer Advisory Panel (NPUAP) combines the last three into Stage 4. Recovering ulcers are cited by their stage at initial observation (e.g., “a healing Stage II pressure ulcer”).

Stage 1: Unbroken red skin (bluish or purplish in dark-skinned individuals) that does not blanch under pressure; may be painful, oddly textured, or abnormally hot or cool.

Stage 2: Swollen skin, blistered or abraded, with damage but no deeper than the dermis.

Stage 3: Ulcer penetrates skin or deeper, reveals deep skin layers; the poor blood supply can defy healing. Subsurface damage may be greater than appears superficially.

Stage 41 The sore extends into muscle.

Stage 5: Muscle is destroyed.

Stage 6: Bone is exposed, damaged, and possibly infected.

Unstageable: dead cells (or eschar) and exudates conceal the degree of damage.

Treatment steps include: debriding damaged tissue, controlling infection, increasing dietary protein (to rebuild tissue), relieving pressure, educating caregivers, and treating the wound. (Rothrock, J. C., Alexander's Care of the Patient in Surgery, 13^{th} ed. (Mosby, 2007)). Intervention in late stages may include skin grafts, negative pressure (i.e., partial vacuum), and antiseptic products (zinc oxide, alcohol wipes) though the latter tend to be ineffective.

Treating wounds in diabetic patients is multidisciplinary because over 100 physiological factors are involved. Total treatment is often costly, and may include dressings (with casts), antibiotics (for staphylococcus, streptococcus and anaerobic strains), debridement, artificial skin, skin transplants, topical human growth factor, high-protein diets, exercise, infrared light therapy (to dilate blood vessels and encourage skin growth) and surgical arterial revascularization. The infections are putrid and often treated with gram quantities of fl(uc)oxacillm, amoxicillin and or metronidazole. Experimental treatments include nitric oxide as a vasodilator (allowing nutrients to reach oxygen-deficient wounds), light therapy, and hyperbaric oxygen therapy. Treatment is often long-term: e.g., hyperbaric therapy commonly evaluates improvements after a year of treatment. Despite widespread use, there is little evidence that therapeutic shoes are effective to treat or prevent diabetic foot ulcers. (S. Spencer, Cochrane Database Syst Rev: CD002302 (2000).) Regranex, a growth factor for diabetes, was deemed the most promising drag by far for treating diabetic ulcers until cancer appeared in 80% of patients in Phase III clinical trials.

Burn care is like pressure ulcer care, including debridement, wound dressings, fluid resuscitation, antibiotics, and skin grafts. Some antibiotics (e.g., silver sulfadiazine) are inadvisable due to prolonging healing times. Biosynthetie dressings may expedite healing but better evidence is needed. Infections, especially by tetanus, are a common problem for burns because the skin is replaced by eschar (moist, proteinaeeous medium lacking blood vessels). Eschar feeds opportunistic infection and allows its proliferation, yet the lack of blood vessels hinders antibiotic distribution and impedes immune ceil migration. Also intermediates in eschar impede the immune response. Thus eschar tissue must often be removed, which can be painful. Poor circulation may also require elevation of burned limbs to prevent edema formation.

For chronic treatment burns are like other surface wounds, but in early treatment burns have special issues: shock, multiple organ dysfunction syndrome, electrolyte imbalance and respiratory distress. Myoglobin and hemoglobin released by damaged muscles and red blood cells in the absence of full fluid, resuscitation cause acute tubular necrosis of the kidneys. Burns also have inflammatory responses, which are local or systemic depending on wound size. And catecholamines released by burned tissue increase heart rate and peripheral vascular resistance.

The ongoing difficulty of healing surface wounds is illustrated by diabetic wounds. Despite its discovered carcinogenicity the drug Becaplermin (Regranex®) has been a leading cicazatrant (drug that stimulates scar tissue); at 20 weeks of treatment it provided a 44% closure rate for diabetic foot ulcers. (See Fang R C, Galiano R D., “A review of becaplermin gel in the treatment of diabetic neuropathic foot ulcers,” Biologies: Targets & Therapy, 2008:2(1) 1-12.) Yet the medical community views that efficacy at best as marginal and unsatisfactory.

As shown for burns, decubitus and diabetic sores, surface wounds remain common, hard to cure, slow to heal, and dangerous in their complications. Thus there is an urgent, ongoing and long-felt need for improved wound care compositions and methods to prevent and treat such conditions mildly, rapidly, efficaciously and inexpensively with minimal scarring.

### BRIEF SUMMARY OF THE INVENTION

The invention provides synergistic compositions and methods to prevent and treat dermal wounds and topical ulcers rapidly. The invention is efficacious and economical in treating patients having cuts, burns or pressure sores, and in treating both non-diabetic patients and patients having Type I and or Type II diabetes. The treatment disinfects and relieves pain while improving topical circulation and restarting the healing cascade.

In a particular exemplary embodiment the invention provides a synergistic medicinal combination for topical use, comprising an extract of Hydrastis canadensis, an extract of a species of the genus Panax and an extract of Aloe barbadensis L., wherein the combination is present in a composition that is essentially free of extract of Passiflora incarnata L.

In another exemplary embodiment the invention provides a method for treating a topical wound of a patient in need thereof, wherein:
a) the wound is present on a mammal in the group consisting of: humans, other primates, canines, felines, rodents, equines, bovines, eervids and similar ruminants, caprines, porcines, ovines, ferrets, rabbits, hares, marsupials and aquatic mammals; and
b) the wound is treated topically with a synergistic medicinal combination comprising an extract of Hydrastis canadensis, an extract of a species of the genus Panax and an extract of Aloe barbadensis L., and wherein the wound is not treated with an extract of Passiflora incarnata L.

In a further exemplary embodiment the invention provides a kit for treatment of topical conditions by a synergistic medicinal combination in patients suffering from topical acute or chronic wounds, comprising the following each of which is essentially free of extract of Passiflora incarnata L.
a) one or more compositions, wherein the compositions in the aggregate comprise:
   i) an extract of Hydrastis canadensis;
   ii) an extract of a species of the genus Panax;
   iii) an extract of Aloe barbadensis L.;
   iv) optionally one or more supplemental substances selected from the following group: antimicrobial substances, oxidizers and skin-enhancing agents;
   v) a formulation base selected from the group consisting of the following: an aqueous solution, a paste, a cream, a hydrogel or other gel, or an ointment; and
b) an applicator device selected from the group consisting of: a syringe, a container equipped with a sprayer nozzle, an applicator designed for viscous materials, and an absorbent wound dressing.

### DETAILED DESCRIPTION OF THE INVENTION

The invention and the claims are described by means of the following terms, for which definitions are provided so that their use herein may be better understood.

### DEFINITIONS

The following definitions are provided to clarify how the terms are used herein.

The term “patient” means an individual in need thereof who is either self-medicated and or medicated by caregivers, either prophylactically and or therapeutically. The term “patient” is not limited to human patients but may include other mammals, such as non-human primates, canines, felines, rodents, equines, bovines, cervids and similar ruminants, caprines, porcines, ovines, ferrets, rabbits, hares, marsupials and aquatic mammals; and may also include patients from other zoological classifications, such as birds, amphibians, reptiles and fish.

The term “topical” as used with respect to the location of a wound or application of a medicinal composition has its usual and ordinary meaning in the medical arts. Regarding administration, the term topical is as opposed to enteral (i.e., in the digestive tract) and parenteral (i.e., injection into the circulatory system). Examples of topical applications include epicutaneous, inhalational (e.g., for wounded mucous membranes), eye drops, ear drops, and surface application to the teeth and or gums. Topical tissues for which the invention is particularly useful include but are not limited to pressures sores on a patient's foot; wounds in oral tissues; and weight-bearing skin positions on which decubitus sores are more likely to form due to a patient's sustained bed rest in particular body positions.

The terms “wound” and “injury” as used are synonymous, and refer to topical sores, topical infections, lacerations, abrasions, contusions, surgical incisions, and other topical wounds, and include but are not limited to the types that are particularly common among diabetics. The term “topical” has its ordinary meaning in the arts of medical treatment. The term “surface” as used with respect to a wound is synonymous with the term topical.

The term “healthy” as used with respect to topical tissue refers to topical tissue in which no sore, wound or infection is evident.

The terms “diabetes” and “diabetic” have their ordinary and common meanings in the medical arts. The terms “Type I,” and “Type II” as used with respect to diabetes have their ordinary and common meanings in the medical arts for diagnosing and treating diabetes. With respect to topical diabetic ulcers “ulcer” has its ordinary and common meaning in medical arts.

The term “burn” as used with respect to a type of wound has its usual and ordinary meaning in the medical arts, particularly with respect to the meanings for first, second and third degree burns, sunburns, chemical burns and radiation burns.

The term “treatment” means medical treatment, and includes both therapeutic and prophylactic treatment, depending upon the context of the use herein. The term “treatment site” means the specific location of a patient's topically exposed tissue that is treated.

The term “therapeutic” as used with respect to treating a wound refers to a treatment for the healing of that wound. Illustrative examples of wounds treated therapeutically according to the invention include sores and pressure ulcers in diabetic patients and alternatively in decubitus.

The term “healing rate” as used with respect to a wound refers to the speed at which that wound is healed. The term “improvement” as used with respect to a wound's healing rate refers to speedier or more complete recovery.

The terms “synergy,” “synergism” and “synergistic” as used with respect to wound healing provided by a combination of herbal extracts used in a composition or medical treatment process means that the wound healing rate improvement provided by the combination exceeds the sum of the wound healing rate improvements provided by individual extracts from the composition when used in isolation from each other.

The terms “prophylactic” and “preventive” as used with respect to a wound are synonymous and refer to a treatment to prevent formation of a wound. Illustrative examples of prophylactic use according to the invention include prevention of the formation of sores and pressure ulcers in diabetic patients.

The term “topical” means exposed tissues of a patient and the tissues that closely underlie them; with, respect to medicinal treatment the term also refers to the application of pharmaceutical composition on such tissues. The term “topical” includes but is not limited to the following exposed areas, where exposure refers not to clothing but to proximity to topically accessible surfaces: skin; exposed surfaces of open wounds such as in cuts, abrasions and lacerations; oral surfaces such as on the gums, tongue, throat and buccal surfaces of the mouth; nasal interior surfaces such as within nostrils; exposed surfaces of eyes and eyelids; exposed surfaces such as within ear canals; and exposed surfaces such as in the anal and genital regions.

The terms “apply,” “applying,” “application” and “treatment” as used with respect to topical surfaces treated by compositions according to the invention refer to treatment of those surfaces with medication and include but are not limited to treatment of unbroken topical tissue as well as treatment of the interior of open wound cavities.

The term “simultaneously) with” as used with respect to topical application of extracts or compounds refers to joint or concurrent application of the indicated substances.

The term “near in time to” as used with respect to topical application of a first substance relative to topical application of another substance refers to separate applications wherein the first is applied earlier than or later than the second substance, and the difference in time is one week or less. In particular embodiments the difference in time is selected from the group consisting of up to: 1 minute, 2 minutes, 5 minutes, 15 minutes, 1 hour, 3 hours, 4 hours, 6, hours, 8 hours, 12 hours, 24 hours, 48 hours, 72 hours, 96 hours, 120 hours, 144 hours, and 168 hours. In particular embodiments the substances are applied within 1 minute to 72 hours of one another; in further embodiments they are applied within 2 minutes to 48 hours of one another; in additional embodiments they are applied within 5 minutes to 24 hours of one another; in still further embodiments they are applied within 15 minutes to 12 hours of one another; in particular embodiments they are applied within 1 to 8 hours of one another; in some embodiments they are applied within 3 to 6 hours of one another; in further embodiments they are applied within 4 hours of one another. The description of on composition as being applied earlier or later than the time of application another composition(s) by a certain amount of time refers to the passage of time between their relative applications.

The term “alternating in time with” as used with respect to topical application of two substances refers to the application near in time to but not simultaneously with one another, wherein the applications separately but near in time are repeated in a cyclical fashion in which each cycle last for a period of hours, days or weeks. According to the invention, altering the relative sequence of application one or more times during this cycling is within the scope of use contemplated under the term “alternating in time with”.

The term “natural product” has its common and ordinary meaning in the arts of organic and medicinal chemistry.

The term “active compound” and “active ingredient” as used herein are synonymous and refer to compounds that provide desired efficacious benefits in medicinal compositions.

The term “plurality” means at least two.

The term “extract” has its normal and ordinary meaning in the art of medicinal herbal compositions, irrespective of the method of extraction, and includes both dry and liquid extracts. Thus the term extract as used herein encompasses tinctures, powders, and aromatic principles, and encompasses extracts obtained by expression, absorption, maceration, distillation, and other means. The term extract also encompasses artificial extracts that have been constituted to mimic the composition of a natural extract, and also encompasses natural extracts that have been reconstituted to mimic an original composition. The term “dried extract” means an extract from which extraction liquids such as water, alcohol or another extraction liquid have been removed. The term “botanical extract” means an extract derived from a plant.

The term “Hydrastis canadensis” refers to species of the genus Hydrastis, and particularly includes H. Canadensis as well as H. palmatum (sometimes designated as Giaucidium palmatum in the literature), but is not so limited.

The term “aloe” refers to member species of the genus Aloe, and in particular includes the species A. barbadensis L. but is not so limited.

The term “ginseng” refers to species of the genus Panax. In a particular embodiment it refers to the species Panax quinquefolius (P. quinquefolius), but is not so limited,.

The term “Passiflora incarnata L.” refers to species of the family Passifloraceae, and includes but is not limited to P. coerula, and P. incarnata.

The term “collagen” has its usual and ordinary meaning in the medical arts, and includes but is not limited to collagen derived from bovine sources. The term collagen as used herein is not limited by the type of collagen, its molecular weight, or its location in the body. The term “hydrolyzed collagen” means collagen that has been hydrolyzed, e.g., by reaction with aqueous base or acid, and may include but is not limited to gelatin. The term “electrospun collagen” refers to collagen that has been subjected to an electrospinning process.

The term “skin enhancing agent” means a substance that enhances dermal health. An illustrative but nonexclusive list of categories of such substances includes moisturizers, humectants, emollients, oils and greases, and other lubricants (to reduce or prevent friction). Moisturizers help to heal or prevent dry skin; illustrative moisturizers are vitamins, hydroxy!acids, retinoids, collagen, elastin, DNA, RNA, lecithin, sodium hyaluronate, sodium passive cutaneous anaphylaxis and ceramides, Humectants absorb moisture or enhance moisture retention by other substances; illustrative examples of humectants include glycerin, sorbitol, urea, alpha hydroxyl acids, sugars, and lactic acid and its salts. Additional natural moisturizing factors (NMF) acting as humectants include amino acids, pyrrolidone carboxyiic acid, lactate, urea, ammonia, uric acid, glucosamine, creatinine, citrate, sodium, potassium, calcium, magnesium, phosphate, chloride, sugar, organic acids, peptides, and others. Emollients make skin soft or supple, or soothe skin or mucous membranes; many illustrative emollients are emulsions, particularly water-in-oil emulsions, and include low-spreading-value emollients (e.g., castor oil, almond oil and oleyl oleate as in facial creams), medium-spreading-value emollients (e.g., octyl dodecanol, hexyl decanol, oleyl alcohol, and decyl oleate as in sun protection creams and oils) and high-spreading-value emollients (e.g., isopropyi stearate, isopropyl palmitate, isopropyf myristate, hexyl laureate, and dioctyl cyclohexane, as in body lotions and creams). Oils and greases act as occlusion agents to prevent water loss from the skin (e.g., lanolin or petrolatum). Lubricants reduce or prevent friction between surfaces; emollients, oils and greases are sometimes lubricants but illustrative other lubricants include, e.g., talc (a solid lubricant) and hydrogels (i.e., hydrophilic lubricants). Non-limiting examples of other skin-enhancing agents include allantoin (which is keratolytic, enhances desquamation of upper layers of dead skin cells, smoothes skin, promotes cell proliferation, soothes, and complexes with irritants and or sensitizing agents), collagen, hyaluronic acid and N-aeetylglucosamine (the latter three of which promote skin health and cosmetic appearance).

The term “composition” means a composition of matter, and optionally may include molecular compounds, plant extracts, insoluble plant matter, and other compositions of matter. The term “pharmaceutical composition” means a composition for use in prevention or treatment of medical conditions. The term “same composition” as used with respect to compounds, extracts or other substances in the same composition refers to the combination of such substances in one composition. The term “different compositions” means compositions that differ as to the identity of one or more extracts or other substances contained therein.

The term “synergistic medicinal combination” means a combination of medicinal substances for which the collective effect in wound healing is synergistic, i.e., the collective effect exceeds the sum of wound healing effects for individual components of the combination.

The term “essentially free of” as used with respect to specific substances refers to compositions that contain at most trace amounts of those substances.

The term “mass ratio” has its usual and ordinary meaning in chemistry and medicinal formulations.

The term “solvent” has its usual and ordinary meaning in the arts of chemistry and pharmaceutical formulation. Illustrative but non-limiting types of solvents contemplated within the invention include any pharmaceutically acceptable solvent among the polar aprotric solvents, and alcohol-based solvents, ketone- and aldehyde-based solvents, halogenated solvents, hydrocarbon-based solvents, and the like.

The term “low alcohol” means a compounds having 1,2,3 or 4 carbons and bearing at least one hydroxyl group. Illustrative but non-limiting examples of low alcohols include methanol, ethanol, 1,2-ethanediol, n-propanol, iso-propanol, 1,2-propanedioi, 1,2,3-propanetriol, cyclopropane!, n-butanol, sec-butanol, tert-butanol, 2-methyl-1-propaneol, 2-methylcyclopropanol, and hydroxymethylcyclopropane.

The terms “oxidizer” and “oxidizing substance” or “oxidizing compound” as used herein are synonymous, and have their ordinary meaning in chemistry. The term as used with respect to the invention contemplates but is not limited to compounds such as hydrogen peroxide and organic peroxides, ozone, potassium nitrate, nitrous oxide, nitric oxide, amyl nitrite, nitroglycerin, sodium nitrite, Tollens reagent, sodium hypochlorite and other hypohalites, chlorite, chlorate, perchlorate and other analogous halogen compounds, iodine and other halogens, and the like.

The term “antimicrobial substance” means a substance or composition thereof that kills and or inhibits the growth of one or more types of microorganisms, i.e., the substance is microbiocidal and or rnicrobiostatic. Such a substance may be selective to kill or inhibit particular types of microorganisms (e.g., a substance that is antifungal but not antibacterial, or like neomycin and its pharmaceutically acceptable salts that is primarily antibacterial) or may be non-selective (such as a disinfectant). The affected microorganisms may be bacteria, fungi, protozoans, or other microorganisms.

The terms “liquid,” “solution,” “foam,” “spray,” “aerosol,” “cream,” “gel,” “hydrogel”, “lotion,” “paste,” and “ointment,” have their usual and ordinary meaning in the field of pharmaceuticals. The term “viscous composition” as used herein with respect to medicinal preparations according to the invention includes but is not limited to gels, pastes, ointments, and other compositions having fluid or fluid-like properties due to being above their freezing points, but which have a low tendency to flow.

The term “dressing” means a bandage, cloth, or other artifact suitable for placing on a wound to protect it or to deliver a pharmaceutical composition. The term “wetted” as used with respect to wound dressings, refers to a liquid or liquid-like composition of the invention on the surface of or within a wound dressing. The term “wet phase” as used with respect to a wetted wound dressing refers to the liquid or viscous portions of the dressing, as opposed to, e.g., fibers comprised in a woven dressing; the wet phase may comprise a liquid, cream, ointment, solution, viscous composition, or other liquid or liquid-like composition according to the invention. The terms “absorbent” and “wettable” as used herein are synonymous and describe a wound dressing denotes that the dressing is able to wick, absorb, adsorb or adhere to a liquid composition according to the invention.

The term “mammal” has its usual and ordinary meaning in biology. The term mammal as used herein includes but is not limited to humans, other primates, canines, felines, rodents, equines, bovines, cervids and similar ruminants, caprines, porcines, ovines, ferrets, rabbits, hares, marsupials, and aquatic mammals.

The term “ulcer size” has its usual and ordinary meaning in dermatology and topical healing. In particular, diminishing of ulcer size as cited herein refers to the relative size of the ulcer during healing compared to its size (typically its circumference but in some cases its depth) at the time when wound treatment began.

The term “pH” has its usual and ordinary meaning in chemistry, and in particular embodiments refers to the pH of fluid exudates from wounds.

The term “granulation” has its usual and ordinary medical meaning for topical healing. In particular, the term granulation as used herein contemplates the formation of perfused, fibrous connective tissue that replaces a fibrin clot in healing wounds, typically but not necessarily growing from the base of the wound and filling it. The term “pink granulation” refers to the characteristic appearance of proliferating granulation cells during that phase in wound healing.

The term “epithelialization” has its usual and ordinary medical meaning for topical healing. In particular, the term epithelialization as used herein contemplates the continuous formation of new epithelial cells at the periphery of the wound, and their migration and riding over one another to form an advancing sheet toward the center of the wound where they meet advancing epithelial cells from the other side, covering underlying new granulation tissue to form a barrier between the wound and the environment. The terms “good” and “regional” as used with respect to epithelialization refer to the appearance of a medically substantial extent of epithelialization during wound healing.

The term “vascularization” has its usual and ordinary medical meaning for topical healing. In particular, the term vascularization as used herein contemplates neovascularization, i.e., the process of angiogenesis, resulting in erythematous tissue due to capillary formation, and occurring concurrently with fibroblast proliferation when endothelial cells migrate to the wound area, the neovascularization being necessary to supply oxygen and nutrients to the new epithelial cells. The term “normal vascularization” refers to a degree of vascularization that is within the range for healthy tissue of the same tissue type.

The term “normal pigmentation” refers to the reappearance of coloration consistent with healthy new unbroken topical tissue for the patient's natural pigmentation.

The term “kit” means a medical kit providing medicinal compositions and application materials to enable practice of the invention. In particular embodiments a kit includes at least one medicinal composition in dry or wet form, optionally for reconstitution, and optionally with different medicinal substances in separate compositions from one another. In further embodiments a kit comprises an oxidizing substance such as hydrogen peroxide, nitroglycerin, nitrogen oxide, sodium hypochlorite, or another oxidizing substance. In additional embodiments a kit optionally includes dispenser hardware such as a syringe, a bottle, a pump, and or a nozzle for liquid or spray, or an applicator designed for viscous materials. In further embodiments a kit includes an absorbent material for application of the compositions of the invention, wherein the absorbent material is a wipe, sponge, or wound dressing capable of absorbing wet medicinal compositions of the invention.

The term “supplemental” as used with respect to an active substance refers to compounds that provide a desired medicinal effect, where the inclusion of that active substance in the treatment composition or treatment protocol is specifically contemplated but is optional.

### Theory of the Invention

Until now there has been no particularly effective treatment for treating diabetic patient' topical wounds. Here the historic difficulties were thought to arise from several factors that required concurrent or near-in-time treatment of each to overcome the difficulties. The invention was guided by the following theoretical considerations, however the invention is not so limited because its exact mechanisms of action have not yet been elucidated. 1) Infection. High blood sugar fosters dangerous fungal infections, e.g., candidiasis; also bacteria and other pathogens may proliferate and destroy tissues under the same conditions. Perspiration in the feet exacerbates this trend. So an antiseptic and or antibiotic is warranted.

2) Poor circulation. High blood sugar makes serum more viscous and slower to circulate, compromising oxygen delivery to subdermal tissues and undermining immune responses. Hence vasodilation and stimulation of local immune responses are desirable.

3) Poor glucose metabolism. Characteristic metabolic imbalances in diabetic patients cause topical tissues in particular to be undernourished. That must be remedied.

4) Edema. Interstitial electrolytes imbalances and high pH are believed to be responsible for edema. The accumulated fluid in interstitial spaces hinders healing by separating epidermal from endodermal tissue. Also, ordinary topical damage tends to produce keloidal or scar tissue. So there is a need to rectify the high pH and interstitial electrolyte imbalance.

5) Inadequate regeneration rate. The sluggish healing rates of diabetic foot ulcers in traditional therapy suggests tissue regeneration rates must be enhanced beyond those attainable by wound cleanliness and blood sugar controls. Thus a skin-healing agent is needed.

6) Nerve damage (Peripheral Neuropathy). Where nociception or other physical insensitivity exists, topical tissues tend to be re-injured unwittingly and without notice. So it would be optimal to include a nerve-healing factor, as well as an analgesic to treat the pain as feeling returns during healing.

That etiological analysis earlier led the inventor to combine botanical extracts in new ways targeted to provide three medicinal functions: modulating the accrual of fluids and tissue (catabolism); controlling infection (antiseptics and antibiotics); and relieving pain (analgesics). It was found surprisingly that a combination of extracts of aloe, ginseng, Hydrastis canadensis and Passiflora incarnata L. and optionally other substances provided a remarkable synergistic improvement in the healing rate of treated patients. See Gorinshteyn, U.S. Pat. No. 8,591,960.

However it has now been found surprisingly that the healing efficacy and pain relief provided by the 3-way combination of aloe, ginseng and Hydrastis canadensis without Passiflora incarnata L. extract is superior to that of the 4-way combination above. This finding was startling because the theory guiding the invention had suggested that Passiflora incarnata L. alone provides three essential benefits that the other three extracts do not: vasodilation; edema prevention (by adjusting concentrations of H⁺, Na⁺ and K³⁰ cations); and reduction of keloid (scar) formation. By contrast, for instance, Hydrastis canadensis extract is known to have vasoconstrictive effects. Moreover this inventor had thought that Passiflora incarnata L. provided a supplemental but important analgesic effect and also possibly a nerve-healing effect. Consequently the finding of similar or better efficacy in the absence of Passiflora incarnata L. was not only unexpected but required a major paradigm shift in the mechanistic theory of the invention. The finding also enables compositions to be prepared at a lower cost, in part because the Passiflora incarnata L. extract may be omitted and in part because solubilization of Passiflora incarnata L. extract at desired concentrations required inclusion of significant amounts of an organic solvent whereas the other extracts are more hydrophilic.

Without being bound by theory, the sections that follow describe the invention composition's design for topical healing in diabetic patients and patients with chronic wounds in general. There is some overlap between the designated components because some constituents in the composition have a plurality of effects.

### Catabolic Component

The catabolic component comprises Hydrastis canadensis extract, assisted by extracts of ginseng, and aloe. Hydrastis canadensis extract is an immunostimulator; and its berberine compounds are also believed here to assist in addressing diabetic delays in wound healing. Aloe extract aids healing and is a transdermal agent and moisturizer. Of these three extracts, ginseng is perhaps the best known for catabolic properties, yet it is believed here that Hydrastis canadensis may be the most important contributor, though the invention is not so limited.

Hydrastis canadensis's natural products such as berberine have been found to have benefits for internal use for treating induced animal diabetes (C. Wang et al., Eur. J. Pharmacol. (August 2009)) as well as human diabetes mellitus (J. M. Wang, et al. Eur. J. Pharmacol. 614 (1-3): 77-83 (July 2009); Y. Zhang et al. (July 2008), J. Clin. Endocrin. Metab., 93 (7): 2559-65. (July 2008)). Benefits reportedly accrue by inducing glycolysis, preventing insulin resistance, and acting like an incretin; the effects of even a little insulin are amplified. Clinical research conducted by the Department of Physiology, Faculty of Medicine, Chinese University of Hong Kong, has suggested that the berberine may also act at both endothelium and underlying vascular smooth muscle to induce vasorelaxation and promote healing.

Such extracts have found safe and wide use for centuries. Native Americans used Hydrastis canadensis extract for health conditions including topical inflammations, debility, dyspepsia, whooping cough, pneumonia, diarrhea, fever, and sour stomach. European settlers of the 18th century used it as a wash for eye inflammations. In the 19th and 20th centuries, folk applications expanded to infections or inflammations of the mucus membranes (e.g., canker sores and sore gums or throat), as well as skin sores, bleeding, menstrual complaints, ulcers, gastritis, colitis, constipation, ringworm, acne, genitourinary infections, thrush, and snake bite.

As for ginseng extract, it has traditionally been regarded as an “adaptogen,” i.e., a tonic herb that tends to normalize and strengthen metabolism and immunity over time. Ginseng is one of a group of herbs that practitioners of traditional Chinese medicine (TCM) use with different actions and indications including stimulation of nerve growth factor and RNA/DNA. synthesis, modulation of neurotransmitter activity and blood sugar levels, and protection against myocardial ischemia. In addition, it may enhance macrophage activity, adrenal hormone production, tissue oxygenation, energy production, and capacity for work and stress. In a controlled study of persons over age 60, this herb increased the ratio of superoxide-dismutase to lipid peroxides. Important known adaptogen constituents of compositions of the invention are triterpenoid saponins. The additional presence of polysaccharides (panaxans A-U), polypeptides, phytosterols (e.g., Betasitosterol), and essential oils and nutrients (e.g.,selenium, Vitamin C, and Vitamins-B) may account for the wide range of effects displayed by these substances.

In TCM, this extract is regarded as a yin or cooling tonic herb that works through the lung, stomach and spleen channels to support the adrenal glands, balance metabolism and increase fluids. It is typically prescribed in cases of stress, asthma, mental fatigue, bronchitis, chronic fevers and weak or infected lungs. Also, numerous native American tribes have used this type of extract for a wide variety of applications, ranging from fever reduction and enhancement of mental faculties and female fertility to geriatric rejuvenation. Panax quinquefolius is particularly useful for the invention but it is not so limited.

The catabolic effects of compositions of the invention may optionally be enhanced by combination therapy, alternation therapy, or some variant, and by supplementing or augmenting compositions of the invention with a complementary vasodilation stimulating compound such as nitroglycerin to improve circulation in the vicinity of a dermal wound. Because of nitroglycerin's systemic potency and affect on other parts of the body, when that compound is used with the present invention care must be taken to avoid applying substantial doses of nitroglycerin within a wound in a way that could lead to its migration into the circulatory system. Fortunately studies to date have found that satisfactory results may be obtained by the invention compositions even without a complementary vasodilation stimulating compound.

### Antiseptic Component

The antiseptic component comprises Hydrastis canadensis extract, optionally assisted by hydrogen peroxide and by silver species. Native Americans used the type of antiseptic extract found in compositions of the invention for a wide range of health conditions including topical inflammations, debility, dyspepsia, whooping cough, pneumonia, diarrhea, fever, and sour stomach. European settlers of the 18th century used it as a wash for eye inflammations. In the 19th and 20th centuries, the folk applications expanded to include inflammations and infections of the mucus membranes (e.g., canker sores and sore gums or throat), skin sores, bleeding, menstrual complaints, ulcers, gastritis, colitis, constipation, ringworm, acne, genitourinary infections, thrush, and snake bite.

Without being bound by theory, the natural products of Hydrastis canadensis in compositions of the invention are thought to be particularly potent. For instance, berberine sulfate is known to be variably but effectively antibiotic against parasites, fungi, mycobacterium, and gram-negative or gram-positive bacteria, including Streptococci, Staphylococci, Tubercle bacillus, Cholera vibrio, E. coli, Trichomonas, Leishmaniasis, Entamoeba, Giardia, Trypanosoma, and Chlamydia. Because the ability to kill gram negative bacteria, e.g., spirochetes, it is a more effective antiseptic than soap. Berberine also exhibits anti-tumor activity against malignancies of the human and rat brain, equivalent to the chemotherapeutic BCNU. This extract also has a vasoconstrictive effect.

Certain optional constituents may be used to enhance the antiseptic properties of compositions of the invention. Antibiotics may be employed such as bacitracin, neomycin, polymixin B and their salts, and such as benzethonium chloride or benzalkonium chloride. Oxidizing compounds such as hydrogen peroxide, hypochlorites, nitrate compounds and the like are antiseptic. Oxidative antiseptics tend to cause mild damage to tissue in open wounds, but for instance hydrogen peroxide is effective at rapidly stopping the slow ooze from blood capillaries that follows abrasions, and at low concentrations is useful in healing. Silver may also be used to amplify antiseptic effects; the aqueous ionic form is less toxic to the body than colloidal silver is. Also, silver ions kill bacteria on contact by a mechanism different from that of the herbal extracts and oxidizing compounds.

The antiseptic effects of the new combination are surprisingly on a par with commercial antibiotics used in surgery. For instance, antibiotic susceptibility testing (AST) was performed overnight by the Kirby-Bauer method in which wafers containing vancomycin (30 ug/mL) or tobramycin (10 ug/mL) were tested independently as standards for inhibition against inoculated lawns of Enterococcae faecal is (29212) and Pseudomonas aeruginosa (27853), respectively, on petri dishes. An aliquot of 50 uL of liquid formulation (containing extracts of Aloe vera, Panax quinquefolius, Hydrastis canadensis and in this case Passiflora incarnata L. (which is not antibiotic), as well as water, ethanol and hydrogen peroxide was tested overnight in a similar fashion. This extract mixture performed as well as tobramycin, approaching the effectiveness of vancomycin, and was far superior to antiseptic effectiveness of either hydrogen peroxide alone or ethanol alone. Indeed, unlike most broad spectrum antibiotics the extract mixture showed better inhibition of gram negative rods than of gram positive cocci. Formulations of the invention that are essentially free of Passiflora incarnata L. are no less effective as antibiotics.

### Analgesic Component

The body releases fluid to interstitial spaces to dilute wound catecholamines; this causes swelling sensed by pain receptors. Formation of keloidal (scar) tissue can also cause pain. The analgesic component employed in the invention arises largely from aloe and Passiflora incarnata L. extracts, but other extracts also play a role. For instance, compounds in Hydrastis canadensis presumably provide pain relief because they suppress proinflammatory cytokines.

The active ingredients of the genus Aloe have been shown to have analgesic and anti-inflammatory effects. There are over 300 varieties of Aloe; of these A. barbadensis L. seems to be the most potent and rich in the active agents, but the invention is not limited to use of that particular species. The synergistic benefits of Aloe observed for the invention are believed here to be due in part to its ability to transport medicinal compounds (including those of the other extracts) through the topical membranes of patients, and to serve as a humectant. However the invention is not so limited. Moreover, certain molecular constituents of Aloe are known to bind to the insulin-like growth factor receptor, thus it is thought here to offer special benefits for healing of tissues in diabetic patients. The exact healing benefits of Aloe are still uncertain, and among the suggested mechanisms in the literature are increasing migration of epithelial cells; maturing collagen more rapidly; reducing inflammation, stimulating epidermal tissue formation, and expressing proliferation markers for immunohistochemical action.

### Healing Effects of Other Variants of the Invention Composition

The use of nitroglycerin as a catabolic supplement is discussed above. Other optional supplements include: other oxidizing compounds (such as hydrogen peroxide to improve dermal microcirculation, and to reduce free radicals at the skin to facilitate healing), silver salts for antimicrobial effect, pH buffers (to keep the wound below pH 8 or 9, unlike its exudates); etc. Some clinical work in the prior art has suggested that healing of diabetic foot ulcers does not occur unless the wound is kept at or below a pH of 8 because nitrogen oxide production does not occur at higher pH's. (Department of Pathological Anatomy, Hospital General de Zona, on the use of L-arginine amino acid as treatment). However patients using treatment by the present invention can begin to heal even when the initial conditions for treatment included a pH of 9.

The compositions herein may be administered by combination in the same formulation, or by alternation of their respective administration at points near in time to one another, or by some variant of these. The various constituent natural products in the extracts are believed to work in conjunction with one another, though it is believed that they can be administered sequentially as opposed to simultaneously when that is convenient or desirable for facilitating other medical objectives. In particular embodiments the difference in time for applying a second extract after applying a first extract may be selected from the group consisting of up to: 1 minute, 2 minutes, 5 minutes, 15 minutes, 1 hour, 3 hours, 4 hours, 6, hours, 8 hours, 12 hours, 24 hours, 48 hours, 72 hours, 96 hours, 120 hours, 144 hours, and 168 hours.

### Synergy

The existence and surprising degree of synergy of the herbs in combination relative to their efficacy individually is highlighted by the fact that the invention compositions achieve complete closure of decubitus and diabetic ulcers as much as 4 times faster than do the best current treatments in allopathic medicine (i.e., synthetic drugs). As discussed in the background section above Becaplermin (Regranex) has been a leading cicazatrant (drug to stimulate scar tissue) despite its carcinogenicity; at 20 weeks of treatment it provided a 44% healing rate for diabetic foot ulcers. (Fang, R. C., and Galiano, R. D., “A review of becaplermin gel in the treatment of diabetic neuropathic foot ulcers,” Biologies: Targets & Therapy, 2008:2(1) 1-12.) The medical community views that efficacy as marginal and unsatisfactory.

For context, in the somewhat less recent state of the art, even after 20 weeks of good wound care only 31% of diabetic neuropathic ulcers were healed, and similarly after 12 weeks of good wound care only 24% of neuropathic ulcers attained complete healing. (See D. J. Margolis, J. Kantor and J. A. Berlin, “Healing of Diabetic Neuropathic Foot Ulcers Receiving Standard Treatment: A Meta-Analysis,” Diabetes Care, 1999; 22(5):692-695). In another study (S. Zirany, H. Schatz and M. Pfohl, “Determmants and estimation of healing times in diabetic foot ulcers,” J Diabetes Complications, 2002; 16(5):327-32) the average time to achieve 95% closure of the wound was 62 to 149 days, and some wounds never closed despite identical care, proper footwear, non-weight-bearing limb support, antibiotics, debridement, tight control of serum glucose levels, and careful monitoring.

Improvement in wound closure time, cellular differentiation, tissue regeneration, keloid avoidance, normal vascularization, emollience and pigmentation set the present invention apart from other medicinal interventions. For instance, within a treatment duration of three to four weeks compositions according to the invention were able to achieve: complete healing of a third-degree burn; 60-65% closure of a Stage III pressure ulcer (that had been complicated by diabetes); and beyond the half-way point for complete healing of a stage II pressure ulcer (that had been complicated by diabetes). Likewise within 1 day a composition according to the invention was able to consistently facilitate complete recovery from redness, swelling and irritation arising from various clinical cosmetic procedures that typically require 3 to 5 days of remediation when treated with conventional skin therapy products.

Mechanistically the synergy of extract combinations according to the invention is surprising for several reasons. Although the literature on alternative medicine includes much advocacy extolling various botanical extracts as panaceas, including as potential treatments for chronic wounds, such claims rely on merely anecdotal evidence and have no proof in the scientific literature. In the course of this research a search was conducted of the Pub Med database of the National Institutes of Health, as well as the U.S. patent database and published patent application database, also U.S.- and foreign-sanctioned pharmacopeias for homeopathic medicine and naturopathic medicine, and also the internet at large. No literature from a reputable publication was found that shows that any of aloe, Hydrastis canadensis or ginseng either (a) offers superior properties over other botanical species in prior art to promote wound healing or (b) offers any benefits for treatment of chronic wounds or severe burns.

In fact, aloe is commonly contraindicated for acute and chronic wounds. See, e.g., Dat, A.D., et al., “Aloe barbadensis L. for treating acute and chronic wounds,” Cochrane Database Syst Rev., (2012 Feb) 15;2:CD008762; and Adams, C. A. Jr. and Deitch, E. A., “Diabetic foot infections,” in Surgical Treatment: Evidence-Based and Problem-Oriented, Holzheimer, R. G., Mannick, J. A., eds., 2001. Contrary to popular belief, according to allopathic medical references aloe actually impedes healing of deep wounds and is also unsuitable for use in chronic wounds. See, e.g., L. Skidmore-Roth (Mosby's Handbook of Herbs & Natural Supplements, 2006 (3^{rd} ed.), p. 29-30. Thus it is apparent that the combination of the invention provides a synergy that surmounts the previous disadvantages of aloe use.

Similarly, Hydrastis canadensis's antibiotic and anti-inflammatory properties do not suggest in isolation that it will provide potent healing synergies. Medical professionals have long used far more potent antibiotics and anti-inflammatory drugs, yet without obtaining clear evidence for faster healing times or more successful wound closure ratios, and in fact finding that among others non-steroidal anti-inflammatory drugs (NSAIDS) impede wound healing. See, e.g., Tables 4 and 5 of B. A. Lipsky and C. Hoey, “Topical antimicrobial therapy for treating chronic wounds,” Clin. Infect. Dis., 2009; 49:1541-1549. See also, Table 1, of the poster by C. Wigston et al. (Cardiff University, 2011), “Pilot study to investigate the effect of drugs and other factors on wound healing in patients attending tertiary wound clinics,” posted at http://www.wounds-uk.com/pdf/cases_{—}10239_{—}141.pdf. See also, N. Broderick, “Understanding Chronic Wound Healing,” The Nurse Practitioner, (2009 Oct.), 34(10): 16-22, at p. 21.

In some ways the invention's synergy is not only unexpected but opposite to expectations. Wound healing is a complex multi-stage process for which the main phases are hemostasis, inflammation, proliferation, and remodeling. Though inflammation is commonly treated to alleviate pain, it is well known that healing's proliferation stage cannot begin if inflammation does not occur. Thus an anti-inflammatory agent such as the berberine compound in Hydrastis canadensis might be projected to slow healing, not accelerate it. See S. Guo and L. A. DiPietro, “Factors affecting wound healing,” J. Dent. Res., 2010 March; 89(3):219-229.

The synergies of extract combinations are also difficult to apprehend credibly in the absence of empirical testing because the prior art does not teach how to find such synergies; they are discovered by trial and error or serendipity. The pitfalls of combinations are numerous: most plant specimens have a prolific and poorly characterized (bio)chemical inventory; that inventory varies widely even within narrow classes of plant compounds; there are many types of cross reactions when extracts from two plants are combined; and plant extracts vary significantly in their composition depending on the species, growth conditions, stage of life, plant part and extraction technique. Consequently just as allopathic doctors are often wary of prescribing new combinations of drugs due to the potential for unknown deleterious drug-drug interactions, naturopathic doctors and others in the botanical medicinal arts tend to prescribe botanical extracts singly and not in combination, parallel or alternation. For instance in some common plants the tannins (a ubiquitous and diverse groups of phytochemicals) are toxic when ingested, and bind with medicinal alkaloids (removing them from bioavailability), precipitate key proteins (removing them from bioavailability), and or bind essential mineral ions. And yet the natural inventory of tannins and their properties in extracts is seldom well known.

Examples of potentially deleterious cross-reactions by other types of phytochemicals include: redox reactions; formation of charge transfer complexes; intercalation in DNA and enzymes; electrostatic self assembly; radical trapping and light induced radical reactions; binding between lectin proteins and sugar moieties; salification of phenolic groups by basic moieties; etc. Those reactions are important but harder to detect than tannin precipitates. Further types of cross-reactions include up- or down-regulation of pathways; allosteric or (antagonistic effects on enzymes or cellular pumps; redox activity at substrates; latex allergy cross-reactions; photochemical liquid crystal effects on membrane transport; self-assembled monolayers affecting surface ab/ad-sorption; etc. Other caveats in combining extracts include: interactions across several physiological pathways; lack of understanding of the physiological basis of medicinal effects; uncertainty as to which compounds are most critical as active ingredients; and cross-reactions of botanicals that cancel or exacerbate each other's effects.

Indeed, after the findings of the earlier invention (in which Passiflora incarnata L. was included and required in the combination, and which previously had been known to have evident but very small benefits for healing topical wounds) the inventor and allopathic specialists inferred that Passiflora incarnata L.'s beneficial effect was in regulation of nitric oxide production for vascular dilation and non-specific COX-2 inhibition for pain management. It now appears that Passiflora incarnata L. extract provides no benefit in the combination and to some extent hinders its efficacy.

### Formulations

Novel combinations of the substances described above have been prepared; the combination of therapeutic benefits in a stable formula, and the ease of dispensing the composition, lends itself well to medical use. The characteristics of a particular blend are described here to provide a representative description, but various permutations on the composition will be enumerated in the examples.

The formula prevents bacterial infection. Variations include hydrogen peroxide for excellent cleansing and antiseptic capabilities in contrast to recent literature, which teaches against use of hydrogen peroxide on the grounds of difficulty in applying a proper dosage. During the invention it seemed possible that peroxide would oxidize medicinal phytochemicals in the mixture, rendering them less efficacious, however deleterious effects were not observed. Also, with, or without peroxide the aqueous mixtures leave no oily residues, so oxygen from air can combine with proteins in the wound and improve blood coagulation. The formula also provides temporary relief of dermal discomfort. The formulas discussed here are believed to enable penetration of the epidermis to reestablish the interstitial electrolyte balance and prevent formation of edema and keloid.

The new compositions also have cosmetic appeal. Unlike many other terpene-containing herbal medicinal formulations, compositions of the invention are a pleasant light green and inconspicuous on skin after application. The formulation's odor is not pronounced. By contrast, chamomile extract, though effective at reversing the effect of some chemical damage to skin, is brown, and it ceases to be effective when its terpene is decolorized for instance by chlorination.

### Indications

A primary medical indication for compositions according to the invention is for treatment and prevention of decubitus ulcers, e.g., in bedridden patients. This is critical during long-term stays such as in nursing home care, hospitalization and rehabilitation.

Another medical indication for compositions according to the invention is for treatment and prevention of diabetic ulcers in the lower extremities of diabetic patients (irrespective of whether they are Type I or Type II), especially the feet.

A third medical indication for compositions according to the invention is treatment and prevention of vascular topical ulcers, such as vascular ulcers, arterial ulcers, and related ulcers.

A further indication medical indication for compositions according to the invention is for treatment of burns, including first, second and third degree burns, sunburns, chemical burns and radiation burns. Where the compositions include an agent such as titanium dioxide or other sun-protective agent, another indication is for prevention of sunburns and UV damage in general.

An additional medical indication for compositions according to the invention is for treatment of cuts, lacerations, abrasions, contusions at topical surfaces, surgical incisions, and other topical wounds arising from mechanical injury to the body.

An alternative medical indication for compositions according to the invention is for treatment and prevention of sores and injuries within the oral cavity, such as on the teeth, gums, tongue, palate, cheeks, and throat. Such indications have been an ongoing problem in the profession, for instance after the installation of titanium dental implants in the gums.

Yet another medical indication for compositions according to the invention is for treatment and prevention of injuries at surfaces within the nasal cavity.

Further medical indications for compositions according to the invention include treatment and prevention of injuries at any other skin, mucous membranes, or other topical surface of the body, including but not limited to lips, eyelids, eyes, ears, ear canals, inhalation passages, genital area, anus, and the like.

### Composition Ranges

The following composition ranges of dried extract weights relative to the whole weight of the liquid or gel formulation are useful in various embodiments of invention compositions.

For Aloe barbadensis L.: in one embodiment 0.15 to 40 weight %; in another embodiment 0.5 to 20 weight %; in a further embodiment 0.75 to 10 weight %; in yet another embodiment 1.0 to 5.0 weight %; in a different embodiment, 1.25 to 2.5 weight %; in an alternative embodiment 1.5 to 2 weight %; in still another embodiment about 1.7 weight %.

For dried extract of a species of the Panax genus: in one embodiment 0.03 to 8 weight %; in another embodiment 0.1 to 4 weight %; in a further embodiment 0.15 to 2 weight %: in yet another embodiment 0.20 to 1.0 weight %; in a different embodiment, 0.25 to 0.50 weight %; in an alternative embodiment 0.30 to 0.40 weight %; in still another about 0.34 weight %.

For dried extract of Hydrastis canadensis: in one embodiment 0.015 to 4 weight %; in another embodiment 0.05 to 2 weight %; in a further embodiment 0.075 to 1 weight %; in yet another embodiment 0.10 to 0.50 weight %; in some embodiments, 0.125 to 0.25 weight %; in an alternative embodiment 0.15 to 0.20 weight %; in still another about 0.17 weight %.

In one embodiment an invention composition comprises one or more organic solvents selected from the group consisting of methanol, ethanol, iso-propanol, n-propanol, tert-butanol, sec-butanol, n-butanol, dimethylsulfoxide, n-methylformamide, 1,2,3-propanetriol, 1,2-propanediol, and 1,2-ethanediol.

In one embodiment water represents 0 to 99.97 volume % of the aggregate volume of the liquid components of a composition according to the invention; in another embodiment it represents 50 to 99 volume %; in a further embodiment it represents 80 to 98 volume %; in yet another embodiment it represents 90 to 97 volume %; in an alternative embodiment it represents 92 to 96 weight %; in still another embodiment it represents about 94 volume % of the aggregate volume of the liquid components of compositions of the invention.

In one embodiment a water-compatible thickening or gelling agent is used such as: gelatin; pectin; agarose; carrageenan; hyaluronan; methylcellulose; hydroxypropyl methylceliulose; a polyacrylamide a silicone hydrogel-forming substance; a cross-linked polymer such as of polyethylene oxide, polyAMPS or polyvinylpyrrolidone; a mixture of a polycationic polymer and polyanionic polymer; compositions comprising polyvinyl alcohol, sodium polyacrylate, or acrylate polymers or copolymers having an abundance of hydrophiiic substituents. In one embodiment the gelation or thickening occurs at room temperature. In another embodiment it occurs upon heating. In a different embodiment thickening or gelation occurs in a particular pH range. In some embodiments a lotion-like texture is formed. In other embodiments a soft gel is formed. In further embodiments a firm gel is formed. In certain embodiments the dry mass of the thickening or gelling agent represents 0.1 to 10% of the final hydrated composition. In other embodiments that dry mass represents 0.3 to 7.5% of the final hydrated composition. In other embodiments that dry mass represents 0.5 to 5% of the final hydrated composition. In additional embodiments that dry mass represents 0.7 to 4% of the final hydrated composition. In particular embodiments that dry mass represents about 1 to 3% of the final hydrated composition.

In one embodiment ethyl alcohol (i.e., when its molecular water content is factored out) represents 0 to 85 volume % of the aggregate volume of the liquid components of a composition according to the invention; in another embodiment it represents 2 to 50 volume %; in a further embodiment it represents 3 to 25 volume %; in yet another embodiment it represents 4 to 10 volume %; in an alternative embodiment it represents 6 to 8 weight %; in still another embodiment it represents about 6.6 volume % of the aggregate volume of the liquid components of compositions of the invention.

In a particular embodiment hydrogen peroxide (i.e., when its molecular water content is factored out) represents 0.03 to 3.0 volume % of the aggregate volume of the liquid components of a composition according to the invention; in another embodiment it represents 0.06 to 1.5volume %; in a further embodiment it represents 0.09 to 0.75 volume %; in yet another embodiment it represents 0.12 to 0.3 volume %; in an alternative embodiment it represents 0.18to 0.24 weight %; in still another embodiment it represents 0.2 volume % of the aggregate volume of liquid components of invention compositions.

In one embodiment an oxidizer is present in a composition according to the invention composition in the range 0-50,000 ppm by mass; in another embodiment it is present in the range 1,000-40,000 ppm; in a different embodiment it is present in the range 2000-30,000 ppm; in a further embodiment it is present in the range 5,000-25,000 ppm; in still another embodiment it is present at about 20,000 ppm; in yet another it is in the range of about 2,000-20,000 ppm. In a particular embodiment the oxidizer is nitroglycerin. In another it is hydrogen peroxide.

In a further particular embodiment a composition according to the invention comprises allantoin. In one embodiment the allantoin represents 0.1 to 15.0 weight % of the composition. In another embodiment it represents 0.5 to 10.0 weight % of the composition. In an additional embodiment it represents 1.0 to 8.0 weight % of the composition. In yet another embodiment it represents 1.5 to 6.0 weight % of the composition. In a further embodiment allantoin represents 2.0 to 4.0 weight % of the composition. In a particular embodiment it represents 2.2 to 3.0 weight %, In another embodiment allantoin represents about 2.5 weight % of the composition.

In another particular embodiment a composition according to the invention comprises collagen. In one embodiment the collagen is bovine collagen. In a further embodiment the collage is bovine type I collagen. In an additional embodiment the collagen is a hydrolyzed collagen. In some embodiments the collagen represents 0.05 to 10.0 weight % of the composition. In further embodiments it represents 0.1 to 5.0 weight % of the composition. In particular embodiments it represents 0.2 to 4.0 weight % of the composition. In some embodiments it represents 0.3 to 2.0 weight % of the composition. In another embodiment collagen represents 0.4 to 1.0 weight %. In one embodiment it represents 0.45 to 0.75 weight. In another embodiment collagen represents about 0.5 weight % of the composition.

In an additional particular embodiment a composition according to the invention comprises hyaluronic acid. In one embodiment the hyaluronic acid represents 0.02 to 5.0 weight % of the composition. In another embodiment it represents 0.05 to 4.0 weight % of the composition. In an additional embodiment it represents 0.075 to 3.0 weight % of the composition. In yet another embodiment it represents 0.1 to 2.0 weight % of the composition. In a further embodiment hyaluronic acid represents 0.12 to 1.0 weight %. In a particular embodiment it represents 0.15 to 0.5 weight % of the composition. In another embodiment hyaluronic acid represents about 0.2 weight % of the composition.

In another particular embodiment a composition according to the invention comprises one or more preservatives. In one embodiment the total preservative represents 0.01 to 5.0 weight % of the composition. In another embodiment it represents 0.03 to 4.0 weight % of the composition. In an additional embodiment it represents 0.05 to 3.0 weight % of the composition. In yet another embodiment it represents 0.07 to 2.0 weight % of the composition. In a further embodiment total preservative represents 0.09 to 1.0 weight % of the composition. In a particular embodiment it represents 0.12 to 0.5 weight %. In another embodiment total preservative represents about 0.2 weight % of the composition.

In a further particular embodiment a composition according to the invention comprises a glucosamine compound represented by glucosamine or N-acetylglucosamine. In one embodiment the glucosamine compound represents 0.1 to 20.0 weight % of the composition. In a further embodiment it represents 2.0 to 15.0 weight % of the composition. In another embodiment it represents 4.0 to 14.0 weight % of the composition. In an additional embodiment it represents 6.0 to 13.0 weight % of the composition. In a particular embodiment the glucosamine compound represents 8.0 to 12.0 weight % of the composition. In a further embodiment it represents 19.0 to 13.0 weight % of the composition. In another embodiment the glucosamine compound represents about 10.0 weight % of the composition.

The examples illustrate preparations and methods, but the invention is not so limited.

### EXAMPLE 1

### Illustrative Preparation of a Reconstituted Extract Composition

The following is an illustrative embodiment of a composition of the present invention, in which a single plant extract is reconstituted in fluid for combination with other ingredients. The upper end of useful ranges for weight % incorporation of the extract is largely determined by its solubility; the lower end is determined by efficacy at that concentration. The choice of organic solvent and the ratios of the liquid ingredients is likewise variable and may depend, for instance, on whether antiseptic effects are desired from the solvent. The specific formulation shown in the table below has been found to be particularly useful but the invention is not so limited.

| | Dry | Exemplary |
|---|---|---|
| Dry Ingredients | Weight Basis | Range |
| Dried extract of Hydrastis canadensis (PE 5%) | 25 g | 3 g-500 g |

| Liquid Ingredients | Volume Basis |
|---|---|
| Hydrogen Peroxide (optional) | 1 liter (3% H₂O₂, 97% water by weight) |
| Ethyl Alcohol (optional) | 1 liter (90% EtOH, 10% H₂O) |
| Water (Purified) | Bring composition's total volume to |
| | 4 gallons |

### EXAMPLE 2A

### Illustrative Preparation of Liquid and Gel Compositions

The following is an illustrative embodiment of a composition of the invention. The upper end of useful ranges for weight % incorporation of each extract is largely determined by their solubility; the lower end is determined by efficacy at that concentration. The choice of organic solvent and ratios of the liquid ingredients is likewise variable. The specific formulation shown below has been found to be particularly useful, but the invention is not so limited.

| | Dry | Exemplary |
|---|---|---|
| Dry Ingredients | Weight Basis | Range |
| Dried extract of Aloe barbadensis L. (pure) | 50 g | 5 g-1000 g |
| Dried extract of Panax quinquefolius root | 50 g | 5 g-1000 g |
| (PE 5%) | | |
| Dried extract of Hydrastis canadensis (PE 5%) | 50 g | 5 g-1000 g |

| | |
|---|---|
| Optional gelling agent | amount sufficient to gel |
| | 4 gallons of water to |
| | desired level of firmness |

| Liquid Ingredients | Volume Basis |
|---|---|
| Hydrogen Peroxide (optional) | 1 liter (3% H₂O₂, 97% water by |
| Ethyl Alcohol (optional) | weight) 1 liter (90% EtOH, 10% H₂O) |
| Water (Purified) | Bring composition's total volume to |
| | 4 gallons |

Standardized dried extracts were obtained in pharmaceutical grade from NatureX (375 Huyler St., South Hackensack, N.J. 07606 (201) 440-5000), with an assay for each batch. The Aloe harbadensis L. extract is quite water soluble. Extracts of Aloe harbadensis L. (estimated 10.0 weight % polysaccharides), Panax quinquefolius (4.0 weight % ginsenosides) and Hydrastis canadensis (3.5-5.0 weight % total alkaloids) in the amounts shown here are approximately fully soluble in 800 ml. of water. The extracts contained a small amount of insoluble fibers from source herbs. The ethanol is optional, but may enhance antiseptic benefit.

Use of a thickening or gelling agent is optional. It may be a natural material such as protein gelatin, guar gum, carrageenan, or another natural gum, or may be a synthetic or quasi-synthetic materials such as polyethylene oxide, methyl cellulose or hydroxypropyi methylcellulose. Many suitable thickening or gelling agents, concentrations and conditions for formulating and gelling aqueous compositions are well known to persons having ordinary skill in the art of gel and thickened solution manufacture. The stability of the gel over a period of several months is an issue and affects the choice of gelling agent where the composition is not prepared freshly for administration to a patient; for instance a hydrogel made with gelatin relapsed into liquid state upon standing for a long period.

In an alternative 30 grams of each extract is used per gallon of liquid. This cures more slowly but allows endothelial tissue to regenerate fully before epithelialization (wound closure).

### EXAMPLE 2B

### Illustrative Preparation of a Cream Composition of the Invention

A 30 g mixture of three dried extracts having the same species identities and ˜1:1:1 dry-mass ratio shown in Example 2A was mixed with and dissolved in 4 kg of Medisca's Versapro™ cream base, further supplemented with 0.5 weight % aliantoin. In this case the cream base also contains aloe extract.

### EXAMPLE 2C

### Illustrative Additional Ingredients

It has been found useful to include additional ingredients in the compositions. In addition to aliantoin, forms of collagen have been found to be beneficial. Bovine collagens are useful. For instance type I bovine hydrolyzed collagen was provided at 0.5 weight % in a cream made according to EXAMPLE 2B for a medicinal formulation. In preliminary studies the cream was extremely effective in regenerating skin in superficial wounds.

Hyaluronic acid is also useful. For instance, a cream from EXAMPLE 2B and containing 0.2 weight % hyaluronic acid is useful for a cosmetic formulation.

Other useful ingredients include preservatives, for instance a non-limiting illustrative preservative offering broad spectrum protection is Lonza's Geogard™ ULTRA product. Geogard™ ULTRA contains a blend of gluconolactone, sodium benzoate and calcium gluconate. This natural product also provides moisturizing, chelating and antioxidant benefits.

### EXAMPLE 3

### Illustrative Treatment Regime

A composition of the invention is used in a manner analogous to a cleansing agent; typically bandages soaked with a liquid or gelled formulation such as from EXAMPLE 2A are left on the affected areas for one to thirty minutes at a time. For instance, clinical studies have used this regimen as often as ordinary procedures would normally require for cleaning bedsores. In an illustrative protocol for a stage 1 bedsore, the liquid composition of EXAMPLE 2A was applied to a clean bandage, which was then attached to the affected area continuously for a period of 20 min. The application was repeated once daily for a total of 5 treatments. Creams such as those of EXAMPLE 2B may be left on the wound and optionally covered with a dressing. Alternatively dressings may be used that are impregnated with the liquid, gel, cream or other carrier for the combined extracts of the invention.

Depending on the wound's severity and responsiveness the extract combination may be applied seven days or more at a frequency of: 1×, 2×, 3×, 4×, 5×, 6×, 8× daily or continuously.

Optionally oxidizing agent in the form of a topical medicinal paste, spray, or other convenient format may be applied to the wound concurrently as an independent formulation, or as a component of an extract, or in alternating fashion with one or more extract, to improve circulation at the site. Pharmaceutical nitroglycerin formulations of about 0.02% concentration are available commercially, and are particularly useful here in concurrent or alternating administration. This same option applies for the descriptions in EXAMPLES 4 and 5 below.

### EXAMPLE 4

### Treating Diabetic Minor Cuts, Wounds, Burns, and Stage I Ulcers

Spray the damaged area liberally with the composition of EXAMPLE 2A to saturate the damaged area and clean the wound. Application should be repeated until bleeding is completely stopped. The cream form (from EXAMPLE 2B) or the gel form of the composition (from EXAMPLE 2A) is then applied and closed with simple non-stick bandages.

### EXAMPLE 5

### Treating Major Wounds, Incisions, Burns, and Stage II or Higher Topical

Ulcers in Diabetic Patients

Bathe wound optionally in liquid composition of EXAMPLE 2A to ensure antiseptic environment and complete saturation. This should provide both sufficient antiseptic activity and substantial pain reduction. When bandaging, apply the gel form (from EXAMPLE 2A) or cream form (from EXAMPLE 2B) to bandage to provide catabolic activity. Expect substantially increased healing rates. Repeat regimen throughout standard bandage rotation procedures.

### EXAMPLE 6

### Clinical Results for Diabetic Ulcers

A severely obese 55 year old male with a long history of diabetes II had stage I diabetic ulceration on both feet. The ulcers were large, black markings, and resembled hematomas but had not yet protruded through the skin. The sores had been treated by medical professionals by various protocols for the prior three years without any benefit. The ulcers were treated daily with a cream form of the invention composition (see EXAMPLE 2B) as described in Example 4. Within 5.5 weeks all visible evidence of the ulcers was gone. The patient is proactively continuing the use daily on his feet for prophylactic care.

### EXAMPLE 7

### Clinical Results for Decubitus

A 79-year-oid male patient with diabetes II had previously had a Stage III pressure ulcer on one foot: that had been treated successfully with a liquid formulation containing a 1:2:1:1 combination by weight of Hydrastis canadensis extract, Panax quinquefolius extract, Passiflora incarnata L. extract, and Aloe barbadensis L. extract, respectively, supplemented by treatment with topical nitroglycerin paste, achieving complete healing in 12 weeks. The patient subsequently developed an equivalent Stage III pressure ulcer on the other foot. That second Stage III pressure ulcer was treated daily by a cream that had been made as in EXAMPLE 2B, where the ulcer was protected by a dressing that was changed daily and each dressing was wetted with the cream before application. Within 8 weeks the ulcer was completely healed (closed with full epithelization). This latter recovery rate significantly surpassed that of the other foot that had been treated with the combination including Passiflora incarnata L. for a comparable Stage III ulcer. The patient is proactively continuing daily use of the composition according to the invention so as to prevent formation of new pressure sores.

### EXAMPLE 8

### Clinical Results for Severe Burns

A 49 year old female patient in good health sustained a localized third degree burn from overheated frying oil. The diameter of the burn was 53 mm and the depth was 11 mm, penetrating the full thickness of the skin and exposing muscle. The burn was treated by applying the cream of Example 2B twice daily for the first week following the injury, and once daily for the following 3.5 weeks to complete epithelization. The only other medicinal substance applied was a topical benzocaine solution applied for pain relief, which was used only during the first several hours after the injury. Within 6 weeks all visible evidence of the burn was gone except for a slight darkness of the skin, and the skin was free of scar tissue.

### EXAMPLE 9

### Clinical Results for Venous Ulcers

Venous ulcers occur on the lower leg. They are an extreme stage of varicose veins, normally require costly treatment to heal, and tend to recur within five years after healing. If left untreated venous ulcers may become infected, leading to cellulitis or gangrene, eventually necessitating amputation of the lower limb. Unfortunately some topical drugs in use for treatment of venous ulcers may cause venous eczema as a side effect. A venous ulcer case was treated by a composition of the invention as follows.

An obese 77 year old female patient with no history of diabetes developed severe cellulitis and later developed venous ulceration. Sharp debridement was performed to remove dead tissue and a hypobaric pressure therapy (wound pump) was employed to stimulate circulation, control exudate and facilitate healing during hospitalization for two weeks. Nevertheless the venous ulceration persisted. The patient was then treated daily with a liquid form of the invention composition as made in EXAMPLE 2A. Within three weeks the wound showed good granulation and 60-65% closure. The treating surgeon and attending nurse deemed that healing rate to be very surprising and much more rapid than could have been expected from prior art medicines. It was also deemed surprising that this topical treatment regenerates not only skin but also well-differentiated vasculature. The treatment protocol was then changed to daily treatment by a cream form of the invention composition as made in EXAMPLE 2B to complete the healing process; within the first 10 days of beginning the cream treatment (i.e., after about 4.5 weeks after the liquid treatment had started the wound had reached 80% closure. Within a total of six weeks from initial treatment with compositions of the invention the wound was essentially completely closed.

### EXAMPLE 10

### Clinical Results for Cosmetic Epidermal Wounds

An open-label clinical cosmetology study was performed on 20 female volunteers by a licensed clinician who had 15 years of experience in clinical cosmetology. Each of the subjects received a facial epidermis removal treatment typically used in spas, consisting of either a chemical peel protocol, a microdermabrasion protocol, or a deep facial cleaning protocol. Each of these three protocols is well known to result in persistent irritation and redness from which a spa client typically requires 3 to 5 days for full recovery. Immediately following each subject's respective protocol her face was treated with the cream formulation of EXAMPLE 2B by gently rubbing the cream onto the skin until fully absorbed.

Approximately 24 hours after treatment with the cream a follow-up evaluation was performed on each subject. Each of the subjects was found to have a complete recovery from the redness and irritation associated with her respective treatment protocol. Moreover unexpectedly the product had also reversed facial acne on subjects who had it, with no new acne appearing. This skin recovery rate was two to three times faster than the average for cosmetic skin therapy products on the market, and according to the clinician the speed of healing was unprecedented. Of the 20 women in the study, all considered the product to be extraordinarily effective; though the product was not on sale 15 subjects proactively requested to buy it.

### EXAMPLE 11

### Clinical Results for Diabetic Foot Wound

An 83-year-old male had been treated diligently for a chronic diabetic ulcer on the heel for a period of 21 months and 18 days. That treatment employed a variety of con vers dona 1wound treatments, including Talymed®, negative pressure therapy, Santyl®, MediHoney®, collagen, and anti-infective. Each method had a modest positive effect for a short period of time, but none was able to jump-start the healing cascade.

At the end of that period the dimensions of the wound were about 3 cm long×2 cm wide×0.2 cm deep, and the ulcer had a surface area of about 7.5 cm². A composition according to the invention prepared as in EXAMPLE 2A was then applied twice daily for two weeks (i.e., Weeks 1 and 2). During this time the surface area size fell to 3.1 cm², a 58% decrease. Due to an interruption in supply of the invention composition the treatment was then halted and replaced for the next two weeks (i.e., Weeks 3 and 4) by use of one of the previous commercially aviaiable wound cleansers. At the end of Week 4 the ulcer had re-expanded to 3,9 cm² surface area, a 25% increase in just two weeks. Treatment with the invention composition was then resumed. By two weeks later (i.e., the end of Week 6), the diabetic ulcers surface area had fallen to 2.0 cm, a 51% reduction in size, and an overall reduction of 75%, Four weeks and tour days after that (i.e., 10.5 weeks after onset of treatment with the invention composition) the ulceration had vanished and the wound was declared entirely healed.

The embodiments of the invention as described herein are merely illustrative and are not exclusive. Numerous additions, variations, derivations, permutations, equivalents, combinations and modifications of the above-described composition and methods will be apparent to persons of ordinary skill in the relevant arts. The invention as described herein contemplates the use of those alternative embodiments without limitation.

I claim:

1) A synergistic medicinal combination for topical use, comprising an extract of Hydrastis Canadensis, an extract of a species of the genus Panax and an extract of Aloe harbadensis L., wherein the combination is present in a composition that is essentially free of extract of Passiflora incarnata L.

2) The combination of claim 1 wherein the composition further comprises a solvent selected from the group consisting of water and low alcohols.

3) The combination of claim 1 wherein the composition is in a form selected from the group consisting of a liquid, a solution, a foam, a spray, an aerosol, a cream, a gel, a hydrogel, a lotion, a paste, and an ointment.

4) The combination of claim 3 wherein the composition is provided in a wet phase of a wetted wound dressing.

5) The combination of claim 1 comprising between 5 and 1000 parts of extract of Hydrastis Canadensis, between 5 and 1000 parts of extract of a species of the genus Panax and between 5 and 1000 parts of extract of Aloe barbadensis L.

6) The combination of claim 5 comprising between 25 and 200 parts of extract of Hydrastis Canadensis, between 25 and 200 parts of extract of Panax quinquefolius and between 25 and 200 parts of extract of Aloe barbadensis L.

7) The combination of claim 1 wherein the respective extracts of Hydrastis canadensis, Panax quinquefolius and Aloe barbadensis L. are provided in a mass ratio of about 1:1:1, respectively.

8) The combination of claim 1 wherein the composition further comprises a substance selected from the group consisting of antimicrobial substances, oxidizers, and skin-enhancing agents.

9) The combination of claim 8, wherein the substance is selected from the group consisting of neomycin and its pharmaceutically acceptable salts, nitroglycerin, hydrogen peroxide, collagen, allantoin, hyaluronic acid and N-acetylglucosamine.

10) The combination of claim 9, wherein the substance is collagen and the collagen is optionally a hydrolyzed collagen and is optionally an electrospun collagen.

11) A method for treating a topical wound of a patient in need thereof, wherein
a) the wound is present on a mammal in the group consisting of: humans, other primates, canines, felines, rodents, equines, bovines, cervids and similar ruminants, caprines, porcines, ovines, ferrets, rabbits, hares, marsupials and aquatic mammals; and
b) the wound is treated topically with a synergistic medicinal combination comprising an extract of Hydrastis canadensis, an extract of a species of the genus Panax and an extract of Aloe harbadensis L., and wherein the wound is not treated with an extract of Passiflora incarnata L.

12) The method of treatment of claim 11 wherein the combination is provided in a single formulation in the form of liquid, a solution, a foam, a spray, an aerosol, a cream, a gel, a hydrogel, a lotion, a paste, and ointment, and wherein the combination is optionally provided in a wet phase of a wetted wound dressing.

13) The method of treatment of claim 11 wherein the combination is used to treat or prevent a pressure sore, wherein the sore may be present on a non-diabetic patient, a type I diabetic patient or a type II diabetic patient.

14) The method of claim 11 wherein for a period of seven days or more the combination is applied with a frequency selected from the group consisting of: daily, twice daily, three times daily, four times daily, five times daily, six times daily, eight times daily, and continuously

15) The method of treatment of claim 11 wherein the combination is used to treat a first, second or third degree burn, a sunburn, a chemical burn, or a radiation burn.

16) The method of treatment of claim 13 wherein the combination is used to treat a dermal wound comprising a laceration, abrasion, contusion, or surgical incision.

17) The method of claim 11, wherein the topical treatment site is selected from the group consisting of: a topical pressure sore on a patient's foot; healthy topical tissue where prophylactic care against potential topical injury is desired; and oral tissue.

18) The method of claim 11 wherein the wound is a topical ulcer and treatment continues until one or more of the following conditions is observed at the topical treatment site: pink granulation; regional epithelialization; pH below 7.0; ulcer size diminished by 80%; normal vascularization; and normal pigmentation.

19) The method of treatment of claim 11 wherein at least one of the extracts is administered alternating in time with and in a separate composition from at least one of the other extracts, and wherein the alternation comprises a difference in time of administration of between one minute and 72 hours.

20) A kit for treatment of topical conditions by a synergistic medicinal combination in patients suffering from topical acute or chronic wounds, comprising the following each of which is essentially free of extract of Passiflora incarnata L.:
a) one or more compositions, wherein the compositions in the aggregate comprise:
i) an extract of Hydrastis canadensis;
ii) an extract of a species of the genus Panax;
iii) an extract of Aloe barbadensis L.;
iv) optionally one or more supplemental substances selected from the following group: antimicrobial substances, oxidizers and skin-enhancing agents;
v) a formulation base selected from the group consisting of the following: an aqueous solution, a paste, a cream, a hydrogel or other gel, or an ointment; and
b) an applicator device selected from the group consisting of: a syringe, a container equipped with a sprayer nozzle, an applicator designed for viscous materials, and an absorbent wound dressing.
